(19) United States
(12) Patent Application Publication
(10) Pub. No.: US 2024/0194290 A1
(43) Pub. Date: Jun. 13, 2024

(54) HIDDEN ANTIBIOTICS IN THE HUMAN PROTEOME

(71) Applicant: THE TRUSTEES OF THE UNIVERSITY OF PENNSYLVANIA, Philadelphia, PA (US)

(72) Inventors: Orlando CRESCENZI, Apice (IT); Eugenio NOTOMISTA, Napoli (IT); César DE LA FUENTA-NUNEZ, Philadelphia, PA (US); Marcelo DER TOROSSIAN TORRES, Philadelphia, PA (US); Marcelo CARDOSO DOS REIS MELO, Philadelphia, PA (US)

(21) Appl. No.: 18/554,531

(22) PCT Filed: Apr. 8, 2022
(86) PCT No.: PCT/US2022/071642

§ 371 Date: Oct. 9, 2023

Related U.S. Application Data
(60) Provisional application No. 63/172,792, filed on Apr. 9, 2021.

(51) Int. Cl. G16B 15/20; A61K 38/00; A61P 31/04; C07K 14/47

(52) U.S. Cl. CPC G16B 15/20; A61P 31/04; C07K 14/47; A61K 38/00

(57) ABSTRACT

Disclosed are methods for computationally identifying candidate antimicrobial peptides by scanning the human protcome, ranking identified sequences according to a scoring system in order to identify the candidate sequences. Also provided are novel candidate antimicrobial peptides, as well as combinations of peptides that exhibit synergistic antimicrobial properties.

### CROSS-REFERENCE TO RELATED APPLICATIONS

The present application claims the benefit of priority to U.S. Provisional Application No. 63/172,792, filed Apr. 9, 2021, the entire contents of which are incorporated herein by reference.

### GOVERNMENT RIGHTS

This invention was made with government support under GM138201 awarded by the National Institutes of Health and HRTRA-21-1-0014 awarded by the Department of Defense. The government has certain rights in the invention.

### TECHNICAL FIELD

The present disclosure pertains to identification and use of novel antimicrobial agents.

### BACKGROUND

According to the Centers for Disease Control and Prevention (CDC), in 2019, 2.8 million antibiotic-resistant infections occurred in the US, leading to approximately 35,000 deaths¹. Such untreatable infections are projected to reach 10 million people per year worldwide, becoming the leading cause of death in our society². This daunting scenario coincides with the lack of innovation in antibiotic discovery. Most antibiotics available today have been used for over 30 years. These drugs often have unintended side effects, readily select for antibiotic resistance, and, in the face of this resistance, are losing effectiveness³. Thus, there is an urgent need to discover new antimicrobial agents to target drug-resistant infections⁴.

### SUMMARY

Disclosed herein are methods for computationally identifying candidate encrypted peptides with predicted antimicrobial activity.

Also disclosed are antimicrobial peptides, peptide, wherein the peptide is any one of SEQ ID NOS: 1-43055.

Also provided are methods of treating a microbial infection comprising administering to a subject in need thereof a pharmaceutically effective amount of a peptide of any one or more of SEQ ID NOS: 1-43055.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

FIG. 1. Discovery of hidden peptide antibiotics encoded in the human proteome. (FIG. 1A) Encrypted peptides were identified within protein sequences from the human proteome using a physicochemical-guided scoring function that took into account the main physicochemical features of AMPs, i.e., length, charge, and hydrophobicity. (FIG. 1B) Normalized abundance of genes encoding different protein classes across the two groups, proteins containing predicted encrypted peptides, and proteins in the entire human genome. The analysis was performed using Panther Proteins Classification system with a false discovery rate cutoff of 0.05.

FIG. 2. Composition and bacteria-targeting properties of encrypted peptides. (FIG. 2A) Amino acid frequency in encrypted peptides compared to known antimicrobial peptides. Amino acid usage was calculated from candidate encrypted peptides from the human proteome and from the DBAASP database. Encrypted peptides had overrepresentation of phenylalanine (F), isoleucine (I), leucine (L), and valine (V) compared to classical AMPs present in the DBAASP database. (FIG. 2B) Antimicrobial activity of the encrypted peptides. Briefly, 10⁶ bacterial cells and serially diluted encrypted peptides (0-128 μmol L⁻¹) were added to a 96-well plate and incubated at 37° C. One day post-treatment, the solution in each well was measured in a microplate reader (600 nm) to check for inhibition of bacteria compared to the untreated controls and presented as a heat map of antimicrobial activities (umol L⁻¹) against 8 pathogenic, 11 gut commensal, and 6 skin commensal bacterial strains. Assays were performed in three independent replicates and heat map OD₆₀₀ values are the arithmetic mean of the replicates in each condition. (FIG. 2C) Predictive power of the physicochemical-based scoring function. The figure shows how the experimentally determined Minimum Inhibitory Concentration (MIC) of selected peptides correlates with their predicted scores. Multiple microbes were grouped in this test, ranging from pathogenic strains to gut and skin commensals. Higher predicted scores correlate with lower MICs (more potent antimicrobial activity).

FIG. 3. Synergy, resistance development and mechanism of action studies of encrypted peptides. (FIG. 3A) Synergistic interactions of encrypted peptides from the CUB domain against A. baumannii, resulting in 100-fold lower concentrations of all the three peptides needed to completely inhibit bacterial growth. (FIG. 3B) Evolution of resistance by A. baumannii to encrypted peptides derived from the CUB domain (red) or polymyxin B (gray) after 30 days of serial passaging in liquid nutrient broth. Peptides and antibiotics were used at sub-inhibitory concentrations. Cells were passaged every 48 h. (FIG. 3C) Schematic showing increased fluorescence resulting from membrane destabilization (left panel—blue) and depolarization (right panel—red) caused by the peptides at their MIC over time. (FIG. 3D) Cytoplasmic membrane depolarization effects of the encrypted peptides against A. baumannii. (FIG. 3E) NPN assays showing the effect of encrypted peptides derived from the CUB domain and natriuretic peptide on permeabilization of the outer membrane of A. baumannii.

FIG. 4. Anti-infective activity and synergistic interactions of encrypted peptides in vivo. (FIG. 4A) Schematic of the skin abscess mouse model used to assess the anti-infective activity of selected encrypted peptides from plasma proteins. (FIG. 4B) SCUB1-SKE25 (25 μmol L⁻¹; 77.9 g mL⁻¹) and SCUB3-MLP22 (25 μmol L⁻¹; 66.9 μg mL⁻¹) showed inhibitory activity, especially when used in combination, against both A. baumannii ATCC19606 and P. aeruginosa PAO1. (FIG. 4C) Mouse weight was monitored throughout the experiment (2 days) to rule out potential toxic effects of the encrypted peptides. (FIG. 4D) Schematic of the neutropenic thigh infection mouse model used to assess the anti-infective activity of selected encrypted peptides from plasma proteins. (FIG. 4E) SCUB1-SKE25 (25 μmol L⁻¹, 77.9 μg mL⁻¹) and SCUB3-MLP22 (25 mmol L⁻¹; 66.9 μg mL⁻¹) either alone or in combination reduced infections caused by A. baumannii ATCC19606 and P. aeruginosa PAO1. (FIG. 4F) Mouse weight was monitored throughout the experiment (8 days) to rule out potential toxic effects of the encrypted peptides.

FIG. 5. Comparison of amino acid usage patterns. The figure shows the frequency of usage of all amino acid residue types in sequences from encrypted peptides (red), the entire human proteome (blue), peptides from DBAASP (green), and peptides from DBAASP (purple) with relative scores above 0.8. The frequency of occurrence of each amino acid residue type shows different residue utilization profiles in each group.

FIG. 6. Distribution of physicochemical properties for AMPs and encrypted peptides. (FIG. 6A) The plot shows the relation between absolute score [as defined in⁹] and peptide length. Circles represent peptides from the DBAASP database and are colored depending on the ratio between charge and length. Black triangles represent the 55 encrypted peptides experimentally tested in this study. (FIG. 6B) The same AMPs from the DBAASP database and 55 encrypted peptides are shown, in this case correlating hydrophobicity and length.

FIG. 7. Antimicrobial activity of encrypted peptides expressed in μg mL⁻¹. Briefly, 10⁶ bacterial cells per mL and serially diluted encrypted peptides (0-250 μg mL⁻¹) were added to a 96-well plate and incubated at 37° C. One day post-treatment, the solution in each well was measured in a microplate reader (600 nm) to check for inhibition of bacterial growth compared to the untreated control group. All data are presented as a heat map of antimicrobial activities (μg mL⁻¹) against 8 pathogenic, 11 gut commensal, and 6 skin commensal bacterial strains. Assays were performed in three independent replicates and heat map OD₆₀₀ values are the arithmetic mean of the replicates in each condition.

FIG. 8. Expression levels of proteins containing encrypted peptides. FIG. 8A: Schematic of the biogeographic region within the human body where proteins containing encrypted peptides are located. Expression levels are displayed in a gradient; organs in blue indicate high expression levels and organs in red, low expression levels. FIG. 8B: Normalized expression level values expressed in log₁₀ intensity based absolute quantification (iBAQ), a commonly used metric for protein abundance⁴⁶.

FIG. 9. Synergy between encrypted peptides found within the same area of the human body. FIG. 9A: Experimental layout of the 96-well plates used for two-way synergy experiments using pairs of encrypted peptides. The following encrypted peptides were used: apelin-36, apelin receptor early endogenous 1, natriuretic peptide, big dynorphin, FIBa-GVV27, vWF-PQR19, SRFP1-KKI32, SRFP1-FAL48, INTb-FTR26, SCUB1-SKE25, SCUB3-KHK26, and SCUB3-MLP22. Briefly, two-fold dilutions ranging from 0 to 50 μmol L⁻¹ of the peptide solutions were plated in 96-well plates and 10⁶ bacterial cells in Nutrient Broth (NB) were added to each well to reach a final volume of 200 μL. The FIC value, which indicates the degree of synergy between two antimicrobial agents against a target microorganism (in this case, P. aeruginosa PAO1) was calculated based on the MICs of the peptides used alone and in combination. FIC index values ≤0.5 indicate synergy; additive effects are captured by 0.5≤FIC≤1; 1≤FIC≤4 indicates indifference; and FIC index ≥4 represents antagonism.

FIG. 10. Membrane permeabilization and depolarization assays for several bacterial strains. FIG. 10A: Outer membrane permeabilization experiments showed that encrypted peptides (SCUB1-SKE25, SCUB3-KHK26 and natriuretic peptide) permeabilized the outer membranes of P. aeruginosa PAO1, B. fragilis ATCC25285, and S. epidermidis as much as they permeabilized the A. baumannii ATCC19606 outer membrane (FIG. 3E). FIG. 10B: Cytoplasmic membrane depolarization assays performed against P. aeruginosa PAO1 and the gut commensal B. fragilis ATCC25285. As shown for A. baumannii ATCC19606 (FIG. 3D), the encrypted peptides did not depolarize the cytoplasmic membrane.

### DETAILED DESCRIPTION OF ILLUSTRATIVE EMBODIMENTS

The presently disclosed inventive subject matter may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a treatment” is a reference to one or more of such treatments and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to +10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.” The phrase “at least about x” is intended to embrace both “about x” and “at least x”. It is also understood that where a parameter range is provided, all integers within that range, and tenths thereof, are also provided by the invention. For example, “2-5 hours” includes 2 hours, 2.1 hours, 2.2 hours, 2.3 hours etc., up to 5 hours.

Publications with potential relevance to the presently disclosed subject matter are cited in the present disclosure using superscripted numerals that correspond to the numbered references that are listed in the present disclosure under the heading “References”, infra.

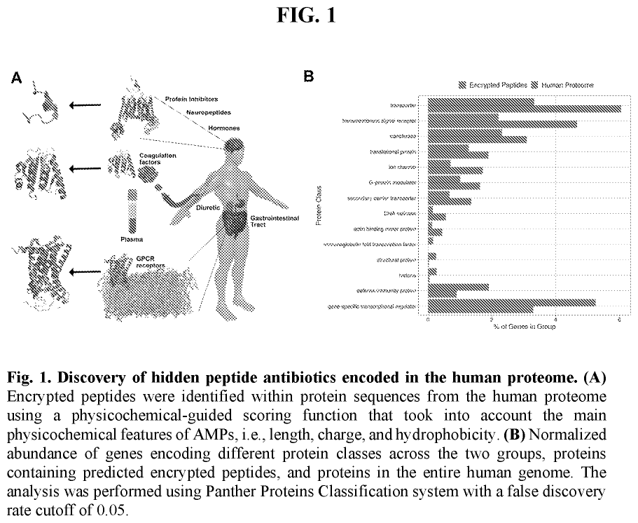

A broad array of computational methods have been developed to expedite drug development, usually focusing on small molecule docking and optimization⁵. However, the application of such methods for antibiotic discovery is still in its infancy⁶. The computer-aided design of antimicrobial peptides (AMPs)^{7,8} has surged as a promising source of new bioactive compounds, which could provide alternatives to conventional antibiotics. AMPs are small molecules (for example, 8 to 50 amino acid residues in length) produced by virtually all living organisms⁹, usually presenting amphipathic and cationic sequences. Here, the present inventors used certain physicochemical features of amino acids to inform a scoring function that comprehensively searched the human proteome for novel antimicrobials (FIG. 1A).

Disclosed herein are methods for computationally identifying candidate encrypted peptides with predicted antimicrobial activity comprising: i) identifying a peptide segment of a protein sequence from the human proteome, the segment being defined by a first sequence window of 5 to 80 amino acids in length, wherein the first residue within the sequence window represents the first residue within the protein sequence; ii) assigning the identified peptide segment a score based on a scoring function that determines mean hydrophobicity, net charge, and length of the peptide, increases the determined mean hydrophobicity by 0.9, increases the determined net charge by 1.1, and multiples all obtained values linearly; iii) identifying a further peptide segment of the protein sequence, the further peptide segment also being defined by the first sequence window length, but wherein the first residue within the sequence window for the further peptide segment represents the second residue within the protein sequence; iv) assigning the further identified peptide segment a score based on said scoring function; v) conducting additional scans of the protein sequence until all segments defined by the sequence window have been identified, and assigning each of the identified segments a score based on the scoring function; vi) ranking all identified segments by score; and, vii) repeating each of steps i)-vi) for all protein sequences from the human proteome; wherein the segments that are ranked among the top 1,000 using the scoring function represent the candidate encrypted peptides for the first sequence window length.

The computational method disclosed herein therefore scans the human genome to find candidate encrypted peptides with predicted antimicrobial activity. The method can utilize one or more of the following: (1) the translated Coding Sequences from the Homo Sapiens genome reconstruction provided by UniProt (publicly available at https://www.uniprot.org/). This is also known as the “human proteome”; (2) a previously developed “scoring function” made public in Pane, K, et al., Journal of Theoretical Biology Volume 419, 21 Apr. 2017, Pages 254-265; and (3) publicly available software development tools and packages in Python. In some embodiments, each of these resources is used pursuant to the present methods.

Preferably, the method scans protein sequences using a “sliding window” technique where, given a window length “L”, every sub-sequence of length “L” is used to calculate a score. For example, in a protein with 100 amino acid, and a window length of 10, the amino acids 1 through 10 will be analyzed as a sub-sequence, then 2 to 11, then 3 to 12, until the window 91 to 100 is verified. In this example, 91 scores for 91 subsequences will be calculated for this protein. The present method scans all proteins in the human proteome with windows of length 5 to 80 (a total of 75 possible window lengths). In some embodiments, the window length is narrower, e.g., 6 to 80, 6 to 75, 6 to 70, 7 to 65, 7 to 60, 7 to 55, 8 to 55, or 8 to 50 residues. In some embodiments, as described more fully below, the methods may use a parallel strategy to significantly accelerate the scanning process. The present method also increases diversity in amino acid sequences by minimizing overlapping sequences and optimizes the search for novel predicted antimicrobial peptides. This method enables the filtering the hundreds of millions of possible sub-sequences in the human proteome down a desired subset. For example, using the present methods, a subset of 43,000 candidate peptide sequences (SEQ ID NOS:1-43000, submitted herewith as Appendix A) was identified.

The present methods may further comprise repeating each of steps i)-vii) for a second sequence window length of 5 to 80 amino acids that is different from the first sequence window length, wherein the segments that are ranked among the top 1,000 using the scoring function represent the candidate encrypted peptides for the second sequence window length. For example, if the sequence window length was 8 residues, then the second sequence window length may be any number from 9 to 50. Thus, the present methods may include repeating each of steps i)-vii) for at least one other possible sequence window length from 5 to 80 amino acids. In some embodiments, the methods comprise repeating each of steps i)-vii) for every other possible sequence window length from 5 to 80 amino acids.

The ranking process of step vi) may exclude from the ranking consecutive segments having a sequence overlap of about 50% or more. More specifically, given a window size of 10 and an initial “base sequence” in a protein (e.g., amino acids 1 through 10), the next selected subsequence of that protein will be the one with the highest score among all 10-residue long sub-sequences that start in residues 6 through 15. If, for example, the sub-sequence between amino acids 8 and 17 has the highest score, that will be the new “base sequence”, and the process starts again.

In some embodiments, the protein sequences from the human proteome includes all protein sequence isoforms from the human proteome. Previous methods did not include protein isoforms.

The methods may include performing step i) with respect to a second protein sequence at substantially the same time as performing step i) with respect to the first protein sequence, using the same sequence window of 5 to 80 amino acids. In other words, a scan of a first protein sequence using a certain window length may be performed in isolation, or alternatively at substantially the same time as scans of a different protein sequence using the same window length. The “parallel strategy” of scanning different proteins at the same time (i.e., simultaneously scanning multiple proteins for each window length), enhances the efficiency of the method. A scan of a first protein sequence that occurs at substantially the same time as a scan of a second protein sequence using the same window length refers to situations in which there is temporal overlap between the respective scans. Accordingly, a scan of a first protein sequence that occurs during a time period that at least partially overlaps the time period during which a scan of a second protein sequence occurs can be said to be at substantially the same time. When there is no overlap between the time period during which a scan of a first protein sequence occurs and the time period during which a scan of a second protein sequence occurs, then the treatments may be described as sequential. Thus, in certain embodiments, scans of respective protein sequences may occur sequentially.

Also provided herein are peptides that were identified using the presently disclosed methods for identifying antimicrobial candidate peptides, wherein said peptide is any one of SEQ ID NOS:1-43055. Also provided herein are methods of treating a microbial infection comprising administering to a subject in need thereof a pharmaceutically effective amount of a peptide of any one of SEQ ID NOS: 1-43055. The present disclosure also provides methods of treating a microbial infection comprising administering to a subject in need thereof a pharmaceutically effective amount of a peptide of any one of SEQ ID NOS:43001-43055. Also disclosed are methods of treating a microbial infection comprising administering to a subject in need thereof a pharmaceutically effective amount of any two or more of SEQ ID NO: 43016, SEQ ID NO: 43015, natriuretic peptide A, SEQ ID NO: 43014, SEQ ID NO: 43024, SEQ ID NO: 43032, SEQ ID NO: 43040, SEQ ID NO: 43039, SEQ ID NO: 43041, SEQ ID NO: 43033, SEQ ID NO: 43034, and SEQ ID NO: 43035. For example, the methods may comprise administering to the subject a peptide of SEQ ID NO: 43033 and a peptide of SEQ ID NO: 43035.

The microbial infection that may be treated according to the present methods may be, for example, viral or bacterial. When the microbial infection is viral, it may include any known viral pathogen. When the microbial infection is bacterial, it may include any known bacterial pathogen. In certain embodiments, the infection may include, Escherichia coli ATCC11775, Pseudomonas aeruginosa PAO1, Pseudomonas aeruginosa PA14, Staphylococcus aureus ATCC12600, Escherichia coli AIG221, E. coli AIG222, Klebsiella pneumoniae ATCC133883, and Acinetobacter baumannii ATCC19606, or any combination thereof.

### EXAMPLES

The present invention is further defined in the following Examples. It should be understood that these examples, while indicating preferred embodiments of the invention, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and conditions.

### Example 1—Identification of Candidate Peptides

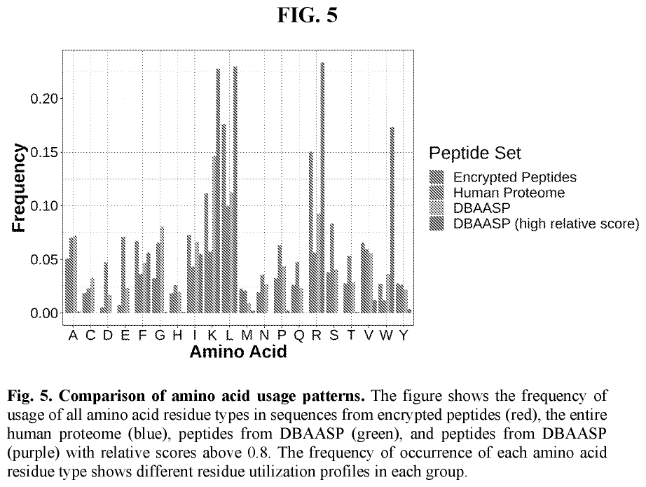

To find novel antibiotic scaffolds, the present inventors intentionally avoided the use of known conserved amino acid sequences (i.e., patterns and motifs) found in previously described AMPs. Instead, physicochemical properties were utilized to guide the discovery approach. This allowed a focus on the balance of key physicochemical features to discover a new region of peptide sequence space populated with antimicrobial peptide sequences (FIG. 5). The search algorithm was based on key physicochemical determinants of AMPs, including net charge, average hydrophobicity, and sequence length, integrated into a fitness function 10 that selects for antimicrobial sequences. Using this scoring function, the entire human proteome, including protein isoforms, was scanned to find peptides ranging from 5 to 80 residues in length, with predicted antimicrobial activity. From the hundreds of millions of possible peptides within 42,361 protein sequences, a total of 43,000 peptide candidates were identified (Appendix A). The Panther Classification System¹¹ was then applied to analyze genes coding for peptides with predicted antimicrobial activity, where an overrepresentation of genes for secreted or membrane-bound proteins was found, as well as an underrepresentation of DNA-bound and gene regulatory proteins (FIG. 1B). This observation supports the hypothesis that encrypted peptides have been evolutionarily selected and maintained in proteins that are exposed to the extracellular environment, where they are more likely to encounter pathogens upon injury and infection.

The search was focused on the most potent and promising encrypted peptides in the human proteome, and the sequences of secreted proteins was scanned to discover 2,603 predicted peptide antibiotics (Table 1).

TABLE 1
Amino acid frequency in encrypted peptides compared to AMPs from DBAASP.

| | | | | | Amino Acid |
|---|---|---|---|---|---|
| Amino | | Frequency (%) | | Ratio | Type Ratio |
| Acid | Amino Acid | Encrypted | Antimicrobial | (Encrypted/ | (Encrypted/ |
| Type | Residue | Peptides | Peptides | Antimicrobial) | Antimicrobial) |
| Aliphatic | Alanine | 5.1 | 7.2 | 0.71 | 1.00 |
| | Glycine | 3.2 | 8.0 | 0.40 | |
| | Isoleucine | 7.3 | 6.6 | 1.10 | |
| | Leucine | 17.6 | 11.2 | 1.56 | |
| | Proline | 3.2 | 4.3 | 0.75 | |
| | Valine | 6.6 | 5.6 | 1.17 | |
| Aromatic | Phenylalanine | 6.7 | 4.7 | 1.43 | 1.16 |
| | Tryptophan | 2.7 | 3.6 | 0.75 | |
| | Tyrosine | 2.8 | 2.2 | 1.28 | |
| Acidic | Aspartic acid | 0.5 | 1.7 | 0.31 | 0.32 |
| | Glutamic acid | 0.8 | 2.4 | 0.33 | |
| Basic | Arginine | 15.0 | 9.3 | 1.62 | 1.08 |
| | Histidine | 1.9 | 2.0 | 0.92 | |
| | Lysine | 11.2 | 14.7 | 0.76 | |
| Polar | Asparagine | 1.9 | 2.7 | 0.71 | 0.942 |
| | Cysteine | 1.9 | 3.3 | 0.58 | |
| | Glutamine | 2.7 | 2.3 | 1.14 | |
| | Methionine | 2.3 | 1.0 | 2.40 | |
| | Serine | 3.8 | 4.1 | 0.93 | |
| | Threonine | 2.8 | 2.9 | 0.96 | |

The identified molecules belonged to two major classes: 1) previously undescribed encrypted peptides derived from proteins with various biological functions, such as plasma proteins, coagulation factors, protein inhibitors, enzymes, and signaling cascade factors; and 2) peptide hormones with well-described putative functions but previously undisclosed antibiotic properties, such as neuropeptides, regulators of G-protein coupled receptors (GPCRs), and diuretic hormones (FIG. 1B). To validate the predictions made by the inventive algorithm, 55 representative peptides (Appendix B, SEQ ID NOS: 43001-43055) were synthesized and characterized in detail; these peptides exhibited a range of predicted scores given by the fitness function and were derived from the cardiovascular, nervous, renal, hematopoietic, and digestive systems.

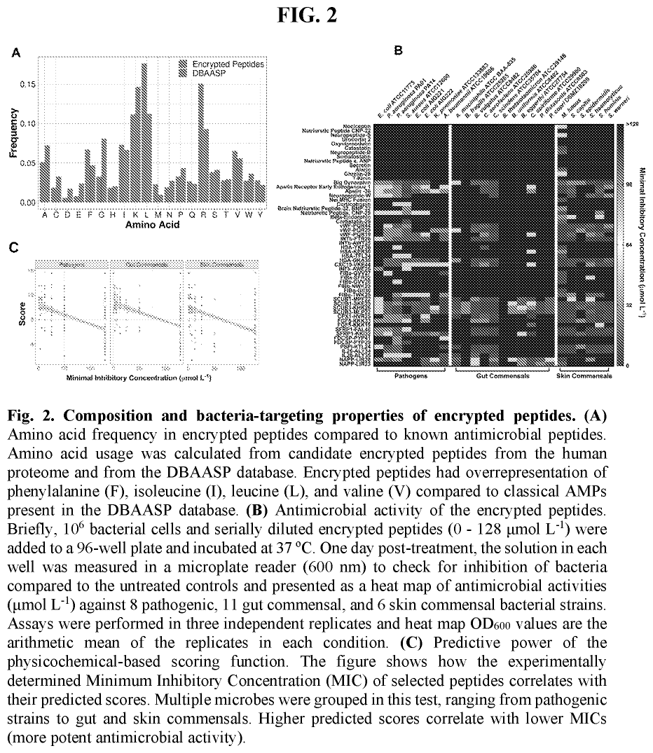

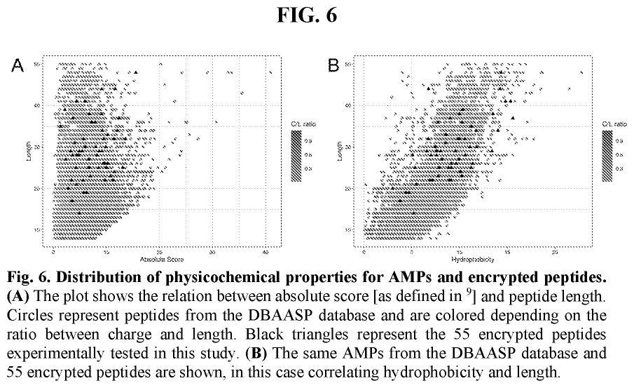

Importantly, the amino acid patterns among the 55 encrypted peptides were significantly different from those of AMPs present in the broadly used DBAASP database¹² (FIG. 2A), even when searches were based on the physicochemical properties displayed by these classical AMPs (FIG. 6). Previously identified AMPs contain motifs and repeats of cationic and hydrophobic amino acid residues, creating an amphipathic structure that plays a key role in their mechanism of action. In contrast, the encrypted peptides we identified displayed heterogeneous sequences that diverged from those of the AMPs. While most AMPs described in the literature¹³ present sequences with 40-60% hydrophobic residues (e.g., leucine, isoleucine, valine, proline, glycine and alanine) and cationic residues (e.g., histidine, lysine and arginine), the encrypted peptides described here displayed a substantially higher content of hydrophobic (16.3%) and basic (8.1%) amino acid residues and a lower content of polar (5.8%) and acidic (68.0%) residues than reported for AMPs, while the content of aliphatic residues was similar (Table 1). For example, out of the nine most common amino acid residues found in AMPs (alanine, arginine, glycine, isoleucine, leucine, lysine, phenylalanine, proline, and valine)¹², four hydrophobic residues (phenylalanine, isoleucine, leucine, and valine) and the basic amino acid residue arginine were more frequently represented in encrypted peptides than in AMPs. AMPs, on the other hand, display small aliphatic and cationic residues (i.e., alanine, glycine, and lysine) at higher frequency than encrypted peptides (FIG. 2A). These data suggest that the encrypted peptides identified and described here may represent a novel class of natural peptide antibiotics, which does not necessarily rely on amphipathic structures but instead consists of arginine-rich, slightly more hydrophobic sequences.

Methods: Human proteome screening for encrypted peptides. To scan the human proteome, all canonical and isoform sequences of Homo sapiens proteins were downloaded from UniProt⁴⁹. A Python script was used to scan all proteins using multiple moving windows with lengths ranging from 5 to 80 residues, leading to millions of peptide sequences. Each sequence was scored using the method described in Pane, et. al.¹⁰, which uses peptide length, charged residues and hydrophobic residues to create a score for the propensity of the peptide to present antimicrobial activity. After the scan, identical peptide sequences were filtered out (even if present in multiple proteins or isoforms) so that only unique sequences were present in the final dataset. Finally, the top 1,000 scoring peptides per sequence length were combined to form a 43,000-long dataset of candidates from across the human genome. An analogous search was carried out to generate a final list of top candidates, but this time, focusing solely on secreted proteins, as those were predicted to provide more biologically relevant encrypted peptides. Peptide Synthesis. The peptides were synthesized using solid-phase peptide synthesis and N-9-fluoromethyloxycarbonyl (Fmoc) strategy. They were purified by high-performance liquid chromatography (HPLC). The peptide purity used in all assays was higher than 95%.

### Example 2—Assessment of Antimicrobial Activity

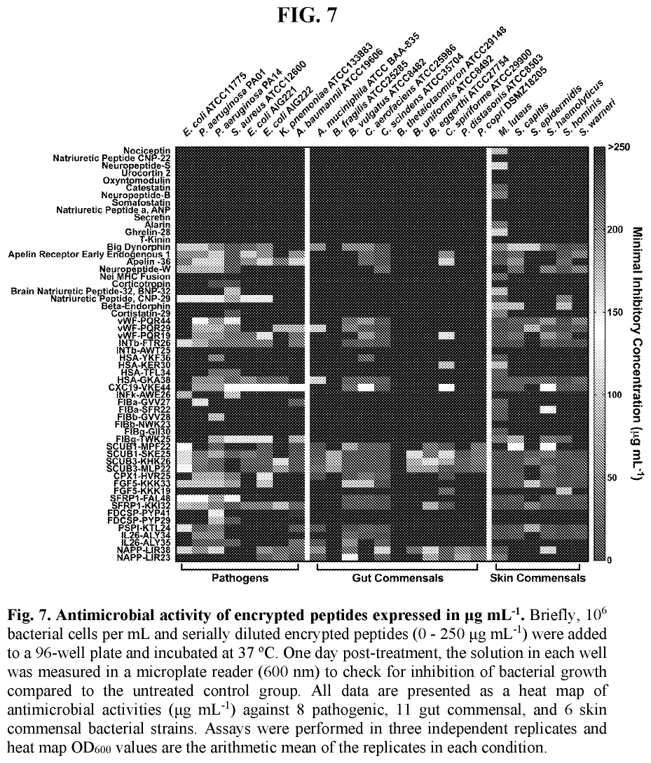

A library composed of 55 encrypted peptides (SEQ ID NOS: 43001-43055—see Appendix B) was synthesized and antimicrobial activity was assessed against 8 clinically relevant pathogens (Escherichia coli ATCC11775, Pseudomonas aeruginosa PAO1, Pseudomonas aeruginosa PA14, Staphylococcus aureus ATCC12600, E. coli AIG221, E. coli AIG222, Klebsiella pneumoniae ATCC133883, and Acinetobacter baumannii ATCC19606—FIG. 2B and FIG. 7), all of which play a major role in infectious diseases and are ranked in the World Health Organization's watchlist 2. Lead encrypted peptides that completely sterilized bacterial cultures (˜10⁶ bacterial cells mL⁻¹) were identified of at least one of these eight pathogens (FIG. 2B and FIG. 7). The majority (63.6%) of the encrypted peptides synthesized and tested displayed antimicrobial activity against pathogens (FIG. 2B and FIG. 7), thus validating the inventive algorithm and indicating the peptides' potential role in host defense, even though they are derived from proteins involved in processes unrelated to the immune system.

In addition to their ability to kill pathogenic organisms, several peptides targeted human commensals from the gut and skin microbiota (FIGS. 2B and 2C), an interesting observation given that some previously described natural peptides are inactive against indigenous microbiota members¹⁴. The 13 most abundant members of the human gut microbiota¹⁵ were exposed to increasing concentrations of the peptides. Species from four different phyla were used to determine susceptibility to the encrypted peptides: Akkermansia muciniphila (Verrucomicrobia); Bacteroides fragilis, Bacteroides thetaiotaomicron, Bacteroides vulgatus, Bacteroides uniformis, Bacteroides eggerthi, Parabacteroidetes distasonis, and Prevotella copri (Bacteroidetes); Collinsella aerofaciens (Actinobacteria); and Clostridium scindens and Clostridium spiroforme (Firmicutes). The peptides displayed low micromolar antimicrobial activity against Gram-positive commensals, and five lead peptides were also able to target A. muciniphila and Bacteroidetes species.

One of the five lead antimicrobial candidates, CPX1-HVR25, derives from the probable carboxypeptidase X1, a protein expressed in many organs that may be involved in cell-cell interactions, collagen binding¹⁶, and regulation of adipogenesis¹⁷; this peptide is classified as a metallocarboxypeptidase based on sequence similarity, although no carboxypeptidase activity has been reported¹⁸. PSPI-KTL24, another peptide with activity against A. muciniphila, a beneficial member of the gut microbiome that has been shown to contribute to obesity, glucose metabolism and intestinal immunity¹⁹, derives from the human plasma serine protease inhibitor and is present throughout the body in the bloodstream. This peptide is responsible for the inactivation of serine proteases by irreversibly binding to their serine activation site and is involved in the regulation of intravascular and extravascular proteolytic activities. In addition, it plays hemostatic roles in blood plasma, acting as a procoagulant and pro-inflammatory factor by inhibiting the anticoagulant activated protein C factor as well as generating the activated protein C factor by thrombin/thrombomodulin complex²⁰. The peptides SCUB1-SKE25, SCUB3-KHK26, and SCUB3-MLP22 also showed potent activity against Gram-negative gut commensals. These are fragments from the terminal portions of two different CUB (complemented C1r/C1s, Uegf, Bmp1) domains, which are structural motifs found in extracellular and plasma membrane-associated proteins. Proteins containing the CUB domains are involved in a diverse range of functions, including developmental patterning, tissue repair, axon guidance and angiogenesis, cell signaling, fertilization, hemostasis, inflammation, neurotransmission, receptor-mediated endocytosis, tumor suppression²¹, and complement activation, which has been shown to contribute to innate immunity by generating antibacterial peptides²². Altogether, the present findings suggest that encrypted peptides may be involved in shaping the gut microbiota, with implications for human health as disruptions in gut commensal communities have been associated with numerous diseases, including obesity, diabetes, inflammatory bowel disease, colitis, cancer and neurodegenerative disorders^{23,24}. Encrypted peptides also targeted skin commensals, including Gram-positive strains isolated from healthy patients, including Micrococcus luteus, Staphylococcus capitis, Staphylococcus epidermidis, Staphylococcus hominis, Staphylococcus haemolyticus, and Staphylococcus warneri (FIG. 2B). Perturbations of the skin microbiota have been associated with conditions such as atopic dermatitis, rosacea, psoriasis, and acne^{25,26}; again, our results revealed novel microbiota-modulating functions of natural peptides. Similar to the gut microbiota experiments, the peptides were active against skin commensals at low micromolar doses (FIG. 2B). To assess the predictive power of our physicochemical-based scoring function, we correlated experimentally determined minimal inhibitory concentrations (MICs) of encrypted peptides with their predicted scores. Generally, the antimicrobial activity of the peptides correlated with their predicted scores against pathogens, and gut and skin commensals (FIG. 2C), and at least 80% of the 55 tested encrypted peptides targeted either pathogens, gut, or skin commensals.

Methods: Bacterial strains and media. The strains used in this study were the pathogens Escherichia coli ATCC11775, Acinetobacter baumannii ATCC19606, Pseudomonas aeruginosa PAO1, Pseudomonas aeruginosa PA14, Staphylococcus aureus ATCC12600, Escherichia coli AIG221, Escherichia coli AIG222 (colistin-resistant strain), Klebsiella pneumoniae ATCC13883, and the gut commensals Akkermansia muciniphila ATCCBAA-635, Bacteriodetes fragilis ATCC25285, Bacteroidetes thetaiotaomicron ATCC29148, Bacteroidetes eggerthi ATCC27754, Bacteroidetes uniformis ATCC8492, Bacteroidetes vulgatus ATCC8482, Parabacteroidetes distasonis ATCC8503, Prevotella copri DSMZ18205, Colinisella aerofaciens ATCC25986, Clostriudium seindens ATCC35704. In addition, we used the following skin commensals in this study: Micrococcus luteus, Staphylococcus capitis, Staphylococucus epidermidis, Staphylococcus haemolyticus, Staphylococcus hominis and Staphylococcus warneri. Pathogenic bacteria were grown and plated on Luria-Bertani (LB) and Pseudomonas Isolation (Pseudomonas aeruginosa strains) agar plates incubated overnight at 37° C., gut microbiome commensals were grown and plated on brain heart infusion (BHI) agar plates supplemented with vitamin K3, hemin and L-cysteine from frozen stocks incubated overnight at 37° C., and skin microbiome commensals were grown in nutrient broth (NB) and plated in tryptic soy broth (TSB) from 4° C. stocks which were incubated overnight at 37° C. Following the incubation period, one isolated colony was transferred to 5 mL of medium (LB, NB or supplemented BHI broth), which were incubated overnight (12-16 h) at 37° C. In the following day, inoculums were prepared by diluting the bacterial overnight solutions 1:100 in 5 mL of the respective media and incubated at 37° C. until logarithmic phase (OD₆₀₀=0.3-0.5). Antibacterial assays. Minimum inhibitory concentrations (MICs) of peptides were determined using the broth microdilution technique in LB, NB and supplemented BHI with an initial inoculum of 5×10⁶ cells in nontreated polystyrene microtiter plates (Corning, USA). Peptides were added to the plate as solutions in LB, NB and supplemented BHI broth in concentrations ranging from 0 to 128 μmol L⁻¹. The MIC was considered as the lowest concentration of peptide that completely inhibited the visible growth (readings were made in a spectrophotometer at 600 nm) of bacteria after 24 h of incubation of the plates at 37° C. All assays were done in three independent replicates.

### Example 3—Synergistic Antimicrobial Activity

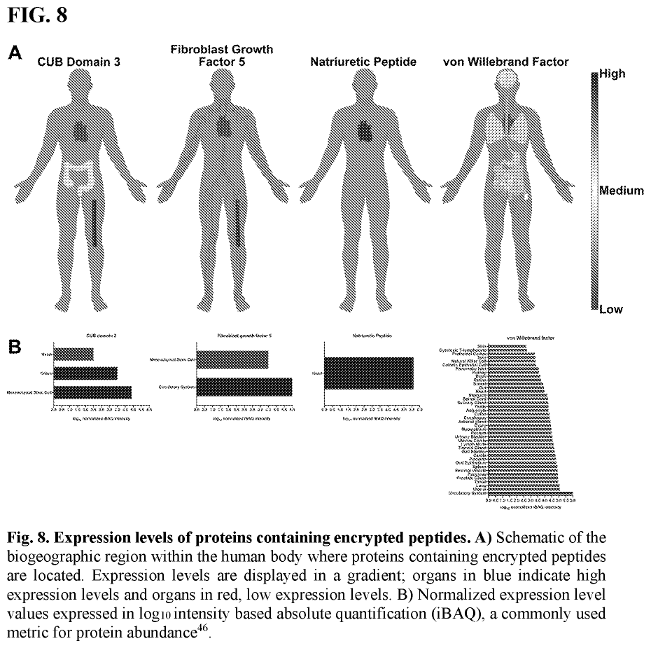

Next, it was investigated whether peptides from the same biogeographical area could synergize to target bacteria at levels that are physiologically relevant (i.e., in most cases from picomolar to millimolar concentrations) (Table 2 and FIG. 8).

TABLE 2
Expression levels in the human body of several proteins
containing 37 of the encrypted peptides studied.

| | | Expression | |
|---|---|---|---|
| | UniProt | levels | Related Encrypted |
| Protein | ID | (pmol L⁻¹)⁴⁶ | peptide(s) |
| Probable carboxypeptidase X1 | Q96SM3 | 10⁶ | CPX1-HVR25 |
| C-X-C motif chemokine 9 | Q07325 | 10⁻⁴ | CXC19-VKE44 |
| Secretin | P09683 | 10⁻⁵ | Secretin |
| Natriuretic peptide A | P01160 | 10⁻⁵ | Natriuretic peptide A |
| Urocortin | P55089 | 10⁻¹ | Urocortin |
| Napsin-A | O96009 | 10⁶ | NAPP-LIR38 |
| | | | NAPP-LIR23 |
| Plasma serine protease inhibitor | P05154 | 10¹¹ | PSPI-KTL24 |
| Follicular dendritic cell secreted | Q8NFU4 | 10⁻⁶ | FDSCP-PYP41 |
| peptide | | | FDSCP-PYP29 |
| Interleukin-26 | Q9NPH9 | 10⁻² | IL26-ALY34 |
| | | | IL26-ALY35 |
| Human serum albumin | P02768 | 10¹⁶ | HSA-YKF36 |
| | | | HSA-KER30 |
| | | | HSA-TFL34 |
| | | | HSA-GKA38 |
| Fibroblast growth factor 5 | P12034 | 10⁻¹ | FGF5-KKK33 |
| | | | FGF5-KKK19 |
| Fibrinogen alpha chain | P02671 | 10¹⁴ | FIBa-GVV27 |
| | | | FIBa-SFR22 |
| Fibrinogen beta chain | P02675 | 10¹⁴ | FIBb-GVV28 |
| | | | FIBb-NWK23 |
| Fibrinogen gamma chain | P02679 | 10¹⁴ | FIBg-GII30 |
| | | | FIBg-TWK25 |
| Interferon beta | P01574 | 10⁻² | INTb-AWT25 |
| | | | INTb-FTR26 |
| Interferon kappa | Q9P0W0 | 10⁰ | INFk-AWE26 |
| Secreted frizzled-related protein 1 | Q8N474 | 10⁸ | SFRP1-FAL48 |
| | | | SFRP1-KKI32 |
| Signal peptide, CUB and EGF- | Q8IX30 | 10⁻² | SCUB3-KHK26 |
| like domain-containing protein 3 | | | SCUB3-MLP22 |
| Signal peptide, CUB and EGF- | Q8IWY4 | 10² | SCUB1-MPF22 |
| like domain-containing protein 1 | | | SCUB1-SKE25 |
| von Willebrand factor | P04275 | 10¹⁰ | vWF-PQR44 |
| | | | vWF-PQR29 |
| | | | vWF-PQR19 |
| Proenkephalin-B | P01213 | 10⁸ | Big dynorphin |

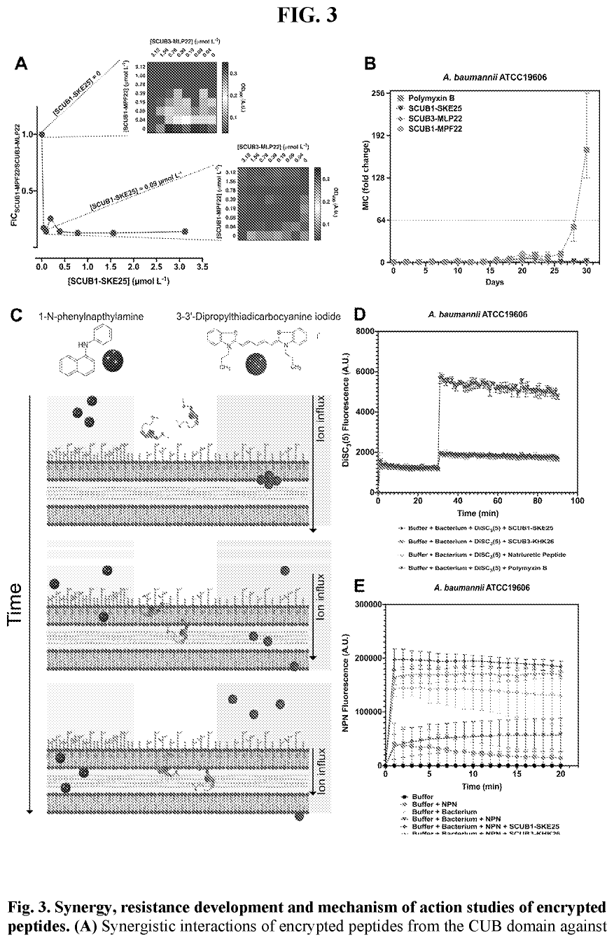

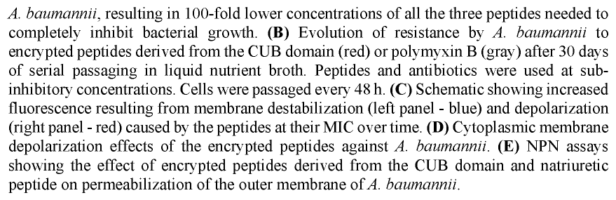

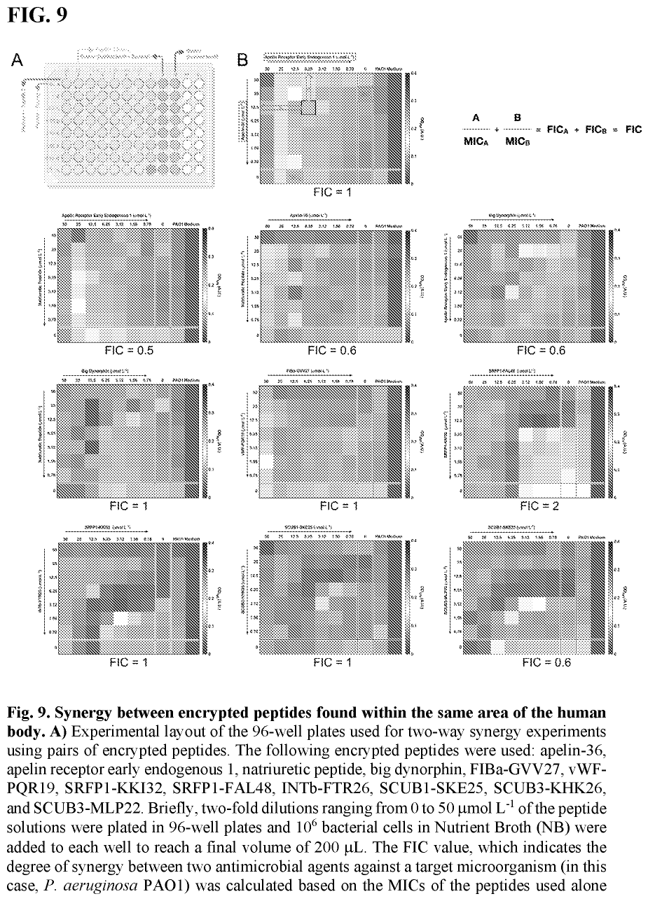

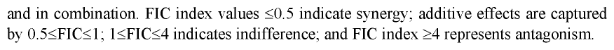

Remarkably, one pair of encrypted peptides synergized to kill pathogens at low micromolar to nanomolar concentrations both in vitro and in animal models (FIG. 3A and FIG. 4B and FIG. 9), displaying activity comparable to, and with even higher potency in some cases than, the most potent venom-derived peptides^{27,28} and defensins from the human immune system²⁹. To investigate synergy, the activity between combinations of 12 peptides was evaluated: apelin receptor early endogenous 1, apelin-36, big dynorphin, natriuretic peptide and encrypted peptides from the coagulation factors, blood glycoproteins, serine proteases, cytokines and the CUB domains. These peptides were assessed for their ability to synergize with each other to inhibit the growth of the Gram-negative pathogenic bacterium Pseudomonas aeruginosa PAO1. The 12 peptides tested in synergy assays were selected on the basis of their potency against P. aeruginosa, an intrinsically resistant bacterium 30 that infects the urinary tract, gastrointestinal tissue, and skin and soft tissues; P. aeruginosa also causes pneumonia and is one of the most common opportunistic pathogens in patients with cystic fibrosis 31. To quantify synergistic interactions between peptides, their fractional inhibitory concentration (FIC) index was determined as previously described^{32,33}. For most of the peptide combinations tested (82%), the peptides interacted additively, with FIC values ranging from 0.6 to 1 (FIG. 9). Highly synergistic interactions (FIC≤0.5) (FIG. 9) were observed between the peptide hormones big dynorphin and apelin receptor early endogenous 1. Big dynorphin is the most potent endogenous opioid peptide, having affinity for k-opioid receptors produced in the brain, and is involved in pain response, control of appetite, circadian rhythms, and temperature regulation³⁴. Apelin receptor early endogenous 1, on the other hand, is responsible for mesendodermal differentiation, blood vessel formation, and heart morphogenesis³⁵. To test synergistic interactions involving more than 2 encrypted peptides derived from the same protein, 3-way synergy experiments were designed and performed. Three representatives were selected from the CUB domains 1 and 3 (SCUB1-SKE25, SCUB1-MPF22, and SCUB3-MLP22), which were active against the Gram-negative pathogen A. baumannii at extremely low concentrations (1.56-3.12 μmol L⁻¹; 4.9-8.2 μg mL⁻¹) comparable to the inhibitory concentrations of standard-of-care antibiotics (e.g., polymyxin B)³⁶. The combination of the 3 peptides increased their individual antimicrobial activity by 100-fold, yielding MIC values of 40-90 nmol L⁻¹ (125-280 ng mL⁻¹) and a combined FIC index of 0.2 (FIG. 3A). Overall, these results are significant because a number of parent proteins containing several of the encrypted peptides reported here are produced physiologically at levels at which the peptides present antimicrobial activity (Table 2 and FIG. 8)³⁷. The ability of these agents to potentiate each other's antibiotic properties further underscores their potential involvement in innate defense.

Methods: Synergy Assays. P. aeruginosa PAO1 and A. baumannii ATCC19606 were chosen for the synergy assays because of their relevance as pathogens that are intrinsically resistant to antimicrobials 30 their ability to infect the urinary tract, gastrointestinal tissue, and skin and soft tissues. After determination of the MIC for each peptide, the most active encrypted peptides against P. aeruginosa and A. baumannii were orthogonally diluted using the microdilution technique with concentrations ranging from 2-fold MIC to 0.03-fold MIC. Plates were incubated for 24 h at 37° C. All assays were done in three independent replicates.

### Example 4—Resistance Assays

In any antibiotic discovery effort, it is crucial to assess how readily the molecules identified select for resistance mechanisms in bacteria, as the emergence of resistance has hampered the antibiotic field since its very inception with the discovery of penicillin in 1928³⁸. To determine whether bacteria become resistant to encrypted peptides, longitudinal resistance assays were performed with peptides derived from the CUB domains 1 and 3 against A. baumannii, using polymyxin B as a control (FIG. 3B). Bacterial cells became highly resistant to polymyxin B after 26 days, when concentrations needed to kill A. baumannii increased by as much as 256-fold (FIG. 3B). Conversely, treatment with the encrypted peptides did not lead to the evolution of spontaneous peptide-resistant A. baumannii mutant cells over the same time period (FIG. 3B). These results identify encrypted peptides as agents that do not readily select for bacterial resistance and point towards a different mechanism of action than that of the AMPs, such as polymyxin B, which have already been characterized.

Methods: Bacterial resistance development assays. The evolution of resistance by A. baumannii cells to encrypted peptides was monitored for 30 days of serial passaging in liquid nutrient broth. The experiment was designed to avoid population extinction. Thus, peptides and polymyxin B, which was used as control, were plated in checkerboard pattern to avoid cross-contamination at concentrations ranging from 8- to 0.25-fold MIC. Bacterial loads of 10⁶ cells were added in each passage and the sub-inhibitory concentrations (0.5-fold MIC) were considered as the first concentration of peptide that inhibited cell growth by ˜50%. Bacteria remaining upon treatment with sub-inhibitory concentrations were re-grown overnight and after each incubation period, a new inoculum of 10⁶ cells was prepared for inoculation of the following passage containing fresh medium and increased doses of the antimicrobial agent. To ensure reproducibility, three parallel experiments with two technical replicates per compound were performed. Cells were passaged every 48 h.

### Example 5—Membrane Studies

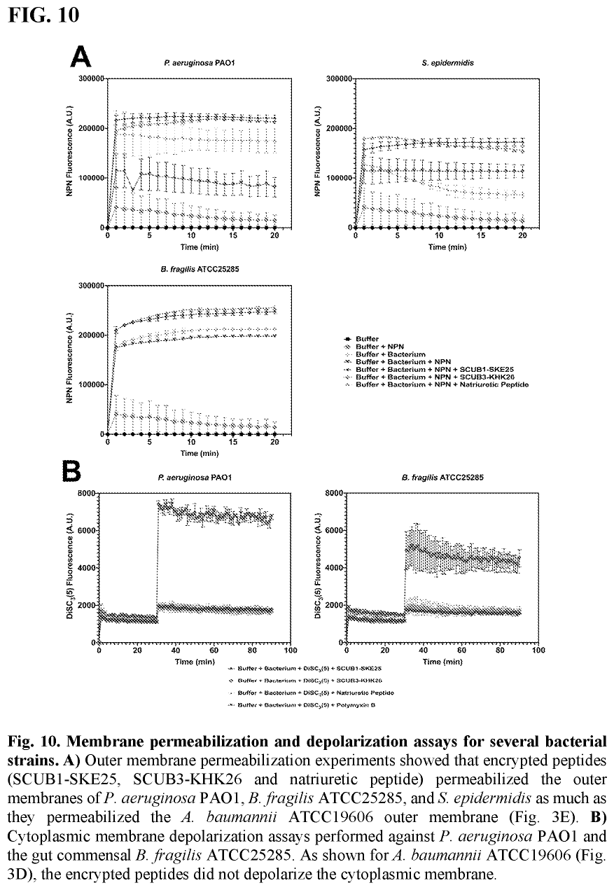

In order to investigate the mode of action of encrypted peptides against bacterial cells, assays were performed to study their effects on the bacterial membrane. First, the ability of encrypted peptides to disrupt and depolarize the bacterial cytoplasmic membrane was assessed (FIG. 3C). 3,3′-dipropylthiadicarbocyanine iodide [DiSC₃(5)] was used, a potentiometric fluorophore that accumulates and aggregates in the cytoplasmic membrane, quenching its fluorescence. Upon imbalances of the cytoplasmic membrane transmembrane potential, the fluorophore migrates to the cytoplasm or the outer environment, generating fluorescence. None of the peptides tested depolarized the cytoplasmic membrane of either pathogenic or commensal bacterial cells to the extent of the positive control group treated with polymyxin B, a well-known depolarizing peptide antibiotic that permeabilizes and disrupts bacterial membranes (FIG. 3D and FIG. 10). These data reveal that encrypted peptides operate via a mechanism that is independent of cytoplasmic membrane depolarization and thus distinct from that of AMPs characterized so far. In order to assess whether encrypted peptides permeabilized the outer membrane, 1-(N-phenylamino)naphthalene (NPN) assays were performed. NPN, a lipophilic dye that fluoresces weakly in aqueous environments but whose fluorescence is greatly increased upon contact with lipidic environments such as bacterial membranes, was added to bacterial solutions (FIG. 3E). NPN does not permeate the bacterial outer membrane unless the membrane is damaged, or its integrity has been compromised. Bacteria exposed to the most active encrypted peptides (i.e., natriuretic peptide, which is responsible for inducing the excretion of sodium by the kidneys, SCUB1-SKE25 and SCUB3-MLP22) at their MIC values (FIG. 3E) emitted increased fluorescence compared to either the untreated control group or to cells treated with the positive control AMP polymyxin B. Thus, encrypted peptides exhibited increased ability to permeabilize the outer membrane compared to conventional AMPs. Overall, these data suggest that the encrypted peptides do not affect the cytoplasmic membrane of bacteria but instead exert their inhibitory effects by permeabilizing the outer membrane.

Methods: Membrane depolarization assays. The cytoplasmic membrane depolarization activity of the peptides was determined by measurements of fluorescence of the membrane potential-sensitive dye, DiSC₃(5). Briefly, A. baumannii ATCC19606, P. aeruginosa PAO1 and B. fragilis ATCC25285 were grown at 37° C. with agitation until they reached mid-log phase (OD₆₀₀=0.5). The cells were then centrifuged and washed twice with washing buffer (20 mmol L⁻¹ glucose, 5 mmol L⁻¹ HEPES, pH 7.2) and re-suspended to an OD₆₀₀ of 0.05 in the same buffer (20 mmol L⁻¹ glucose, 5 mmol L⁻¹ HEPES, pH 7.2) but containing 0.1 mol L⁻¹ KCl. Thereafter, the cells (100 μL) were incubated for 15 min with 20 nmol L⁻¹ of DiSC₃(5) until a stable reduction of fluorescence was achieved, indicating the incorporation of the dye into the bacterial membrane. Membrane depolarization was then monitored by observing the change in the fluorescence emission intensity of the membrane potential-sensitive dye, DiSC₃(5) (λₑₓ=622 nm, λₑₘ=670 nm), after the addition of peptides (100 μL solution at MIC values). Membrane permeabilization assay. The membrane permeability of the peptides was determined by using the N-phenyl-1-napthylamine (NPN) uptake assay. A. baumannii ATCC19606, P. aeruginosa PAO1, S. epidermidis and B. fragilis ATCC25285 were grown to an OD₆₀₀ of 0.4, centrifuged (10,000 rpm at 4° ° C. for 10 min), washed and resuspended in buffer (5 mmol L⁻¹ HEPES, 5 mmol L⁻¹ glucose, pH 7.4). 4 μL of NPN solution (0.5 mmol L⁻¹-working concentration of 10 μmol L⁻¹ after dilutions) was added to 100 μL of the bacterial solution in a white 96-wells plate. The background fluorescence was recorded at λₑₓ=350 nm and λₑₘ=420 nm. Peptides solution in water (100 μL solution at their MIC values) were added to the 96-wells plate, and fluorescence was recorded as a function of time until no further increase in fluorescence was observed (20 min).

### Example 6—In Vivo Models

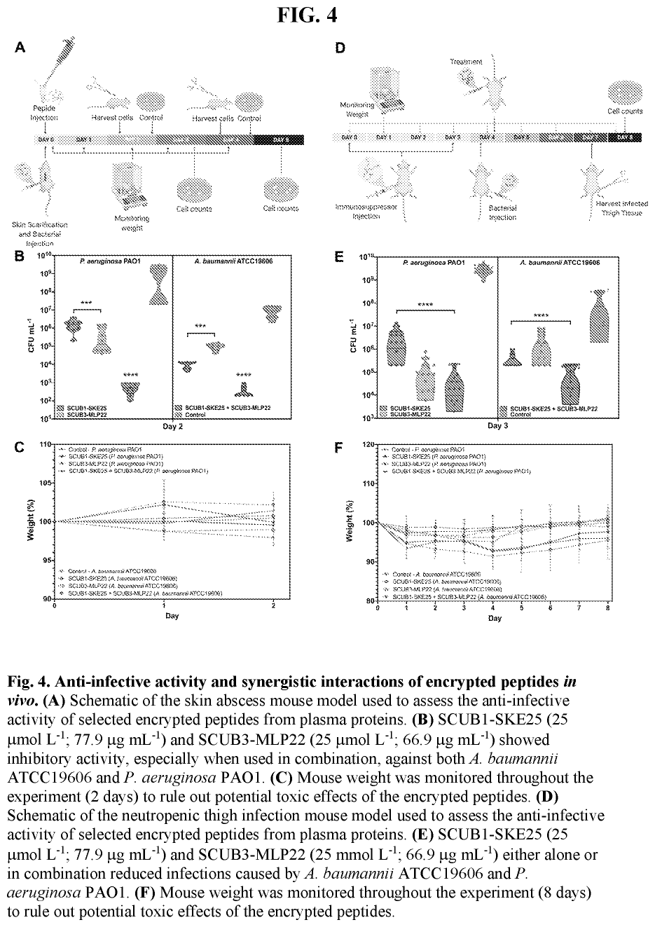

To test whether the peptides retain their antimicrobial activity in a complex living system, their properties in two mouse models were assessed. The most active encrypted peptides, SCUB1-SKE25 and SCUB3-MLP22, were selected. Since these peptides are mostly found in plasma, a relevant skin abscess mouse model was used to test their anti-infective activity (FIG. 4A). Mice were infected with bacterial loads of 10⁵ and 10⁶ cells in 20 μL of the clinically relevant Gram-negative pathogens P. aeruginosa and A. baumannii, respectively (FIG. 4B). A single dose of each encrypted peptide (25 μmol L⁻¹, 77.9 μg mL⁻¹ and 66.9 μg mL⁻¹) delivered to the infected area markedly reduced the bacterial load by 3 orders of magnitude, highlighting the anti-infective potential of these agents. In order to validate the synergistic effects obtained in vitro (FIG. 3D and FIG. 9), infected mice were treated with combinations of encrypted peptides (FIG. 4B). Treatment with a combination of SCUB1-SKE25 and SCUB3-MLP22 significantly decreased bacterial counts by 5 and 6 orders of magnitude for A. baumannii and P. aeruginosa, respectively (FIG. 4B). No damage or deleterious effects exerted by the peptides were detected in the mice in any of our experiments. Of note, the anti-infective activity displayed by the encrypted peptides was comparable to that of some of the most potent antimicrobial peptides described to date, such as polybia-CP and PaDBS1R6^{28,39-42}.

Next, the efficacy of the same encrypted peptides (SCUB1-SKE25 and SCUB3-MLP22) was assessed in monotherapy and in combination therapy in a murine thigh infection model (FIG. 4D). This preclinical model is sensitive, standardized, and widely used for assessing antibiotic translatability^{43,44}. Briefly, mice were rendered neutropenic by cyclophosphamide treatment prior to intramuscular infection with 10⁶ cells mL⁻¹ of the bacterial pathogens P. aeruginosa and A. baumannii (FIG. 4E). Either a single dose of each encrypted peptide (at 25 μmol L⁻¹) or their combination (also at 25 μmol L⁻¹) were delivered to the infected site via intramuscular administration. Three days post-treatment, on average the peptides reduced the bacterial load of P. aeruginosa and A. baumannii by 4 and 2 orders of magnitude, respectively, compared to untreated mice. Compared to untreated controls, treatment with SCUB1-SKE25 and SCUB3-MLP22 significantly decreased bacterial counts of both pathogens by 3 orders of magnitude, and combination treatment reduced by 7 and 5 orders of magnitude infections caused by P. aeruginosa and A. baumannii, respectively (FIG. 4E). Importantly, no significant changes in weight, a proxy for toxicity, were observed in our experiments (FIG. 4C and FIG. 4F), thus further demonstrating the lack of toxicity of the encrypted peptides in relevant mouse models. These in vivo results demonstrate the antibiotic and synergistic properties of encrypted peptides under physiological conditions and strongly suggest that the peptides constitute excellent candidates for the development of antibacterial agents.

Methods: Skin abscess infection mouse model. A. baumannii ATCC19606 and P. aeruginosa strain PAO1 were used to infect the murine skin. Briefly, bacteria were grown in tryptic soy broth (TSB) medium. Subsequently, cells were washed twice with sterile PBS (pH 7.4, 13,000 rpm for 1 min), and resuspended to a final concentration of 1×10⁵ and 1×10⁶ CFU/20 μL for A. baumannii and P. aeruginosa, respectively. Female CD-1 mice (six-weeks old) were anesthetized with isoflurane and had their backs shaved and a superficial linear skin abrasion was made with a needle in order to damage the stratum corneum and upper-layer of the epidermis. An aliquot of 20 μL containing the bacterial load in PBS was inoculated over each defined area containing the scratch with a pipette tip. One hour after the infection, peptides at their MIC value were administered to the infected area. Animals were euthanized and the area of scarified skin was excised two- and four-days post-infection, homogenized using a bead beater for 20 minutes (25 Hz), and 10-fold serially diluted for CFU quantification. Two independent experiments were performed with 8 mice per group in each condition. Neutropenic thigh infection mouse model. Two doses of cyclophosphamide (150 mg Kg-1) applied intraperitoneally with an interval of 3 days were used to render the mice neutropenic. One day later, the mice were infected intramuscularly in their right thigh with a bacterial load of 10⁶ CFU mL⁻¹ of the pathogens A. baumannii ATCC19606 and P. aeruginosa PAO1, which were grown in TSB, washed twice with PBS and resuspended to the desired concentration. Two hours later, peptides were administered in the infection site. Prior to each injection, mice were anesthetized with isoflurane and monitored (respiratory rate and pedal reflexes). Next, we monitored the formation of the abscess and euthanized the mice. The infected area was excised three-days post-infection, homogenized using a bead beater for 20 min (25 Hz), and 10-fold serially diluted for CFU quantification. One experiment was performed using 16 mice per group in each condition (untreated controls, mice treated with a single peptide and mice treated with combination therapy of two peptides).

Although several approaches have been previously taken⁴⁵⁻⁴⁷ to identify novel molecules in the human microbiota and soil bacteria⁴⁸, to date, a systematic study exploring the human body as a potential source of drugs has not been performed. Here, reported is the discovery of 2,603 previously undescribed antibiotics encrypted within the human proteome. At least 55 of these agents, tested as described herein, were found to target both pathogens and commensals, to operate by permeabilizing the bacterial outer membrane, to display synergistic interactions that potentiate their activity, and to effectively reduce infections in a relevant rodent model. It has been shown herein that encrypted peptides differ from conventional AMPs because they: 1) possess a different amino acid frequency profile (FIG. 2A); and 2) target commensals (FIG. 2B-2C). These results point to the proteome as a previously untapped source of novel antibiotics and reveal the multifunctional nature of numerous proteins that were traditionally thought to have only a single biological function. Because the presently disclosed approach computationally identifies antibiotics already optimized by nature and produced in our own bodies, it is expected that they will serve as excellent candidates for antibiotic development. The data presented also suggest connections between host defense and other systems in the body that were previously unrecognized as arms of host immunity, such as the nervous, endocrine, digestive, and cardiovascular systems. More broadly, it is believed that the existence of proteins with multiple functions, enabled by encrypted fragments, reflects an evolutionary mechanism that expanded protein capabilities while minimizing genomic expansion. This reduced the number of protein coding genes that perform all the functionalities necessary to operate and defend the human body.

### REFERENCES

The following references may be relevant to the present disclosure:
1. Centers for Disease Control and Prevention. 2019 Antimicrobial Resistant Threats Report. (2019).
2. World Health Organization. Antimicrobial resistance: Global Health Report on Surveillance. Bull. World Health Organ. 1-256 (2014). doi:10.1007/s13312-014-0374-3
3. Lepore, C., Silver, L., Theuretzbacher, U., Thomas, J. & Visi, D. The small-molecule antibiotics pipeline: 2014-2018. Nat. Rev. Drug Discov. (2019). doi:10.1038/d41573-019-00130-8
4. de la Fuente-Nunez, C., Torres, M. D., Mojica, F. J. & Lu, T. K. Next-generation precision antimicrobials: towards personalized treatment of infectious diseases. Curr. Opin. Microbiol. 37, 95-102 (2017).
5. Torres, P. H. M., Sodero, A. C. R., Jofily, P. & Silva-Jr, F. P. Key Topics in Molecular Docking for Drug Design. Int. J. Mol. Sci. 20, 4574 (2019).
6. Torres, M. D. T., Cao, J., Franco, O. L., Lu, T. K. & de la Fuente-Nunez, C. Synthetic Biology and Computer-Based Frameworks for Antimicrobial Peptide Discovery. ACS Nano 15, 2143-2164 (2021).
7. Torres, M. D. T. & de la Fuente-Nunez, C. Toward computer-made artificial antibiotics. Curr. Opin. Microbiol. 51, 30-38 (2019).
8. Porto, W. F. et al. In silico optimization of a guava antimicrobial peptide enables combinatorial exploration for peptide design. Nat. Commun. 9, (2018).
9. Zasloff, M. Antimicrobial peptides of multicellular organisms. Nature 415, 389-395 (2002).
10. Pane, K. et al. Antimicrobial potency of cationic antimicrobial peptides can be predicted from their amino acid composition: Application to the detection of “cryptic” antimicrobial peptides. J. Theor. Biol. 419, 254-265 (2017).
11. Mi, H., Muruganujan, A., Ebert, D., Huang, X. & Thomas, P. D. PANTHER version 14: more genomes, a new PANTHER GO-slim and improvements in enrichment analysis tools. Nucleic Acids Res. 47, D419-D426 (2019).
12. Pirtskhalava, M. et al. DBAASP v.2: An enhanced database of structure and antimicrobial/cytotoxic activity of natural and synthetic peptides. Nucleic Acids Res. 44, D1104-D1112 (2016).
13. Kang, X. et al. DRAMP 2.0, an updated data repository of antimicrobial peptides. Sci. Data 6, 148 (2019).
14. Cullen, T. W. et al. Antimicrobial peptide resistance mediates resilience of prominent gut commensals during inflammation. Science (80-.). 347, 170-175 (2015).
15. Almeida, A. et al. A new genomic blueprint of the human gut microbiota. Nature 568, 499-504 (2019).
16. Kim, Y.-H., O'Neill, H. M. & Whitehead, J. P. Carboxypeptidase X-1 (CPX-1) is a secreted collagen-binding glycoprotein. Biochem. Biophys. Res. Commun. 468, 894-899 (2015).
17. Kim, Y. et al. Identification of carboxypeptidase X (CPX)-1 as a positive regulator of adipogenesis. FASEB J. 30, 2528-2540 (2016).
18. Lei, Y., Xin, X., Morgan, D., Pintar, J. E. & Fricker, L. D. Identification of Mouse CPX-1, a Novel Member of the Metallocarboxypeptidase Gene Family with Highest Similarity to CPX-2. DNA Cell Biol. 18, 175-185 (1999).
19. Cani, P. D. & de Vos, W. M. Next-Generation Beneficial Microbes: The Case of Akkermansia muciniphila. Front. Microbiol. 8, 1765 (2017).
20. Brenner, A. V. et al. Common Single Nucleotide Polymorphisms in Genes Related to Immune Function and Risk of Papillary Thyroid Cancer. PLOS One 8, e57243 (2013).
21. Bork, P. & Beckmann, G. The CUB Domain. J. Mol. Biol. 231, 539-545 (1993).
22. Nordahl, E. A. et al. Activation of the complement system generates antibacterial peptides. Proc. Natl. Acad. Sci. 101, 16879-16884 (2004).
23. Durack, J. & Lynch, S. V. The gut microbiome: Relationships with disease and opportunities for therapy. J. Exp. Med. 216, 20-40 (2019).
24. Shreiner, A. B., Kao, J. Y. & Young, V. B. The gut microbiome in health and in disease. Curr. Opin. Gastroenterol. 31, 69-75 (2015).
25. Grice, E. The skin microbiome: potential for novel diagnostic and therapeutic approaches to cutaneous disease. Semin. Cutan. Med. Surg. 33, 98-103 (2014).
26. Sanford, J. A. & Gallo, R. L. Functions of the skin microbiota in health and disease. Semin. Immunol. 25, 370-377 (2013).
27. Robinson, S. D. et al. A comprehensive portrait of the venom of the giant red bull ant, Myrmecia gulosa, reveals a hyperdiverse hymenopteran toxin gene family. Sci. Adv. 4, eaau4640 (2018).
28. Torres, M. D. T. et al. Structure-function-guided exploration of the antimicrobial peptide polybia-CP identifies activity determinants and generates synthetic therapeutic candidates. Commun. Biol. 1, 221 (2018).
29. Magana, M. et al. The value of antimicrobial peptides in the age of resistance. Lancet Infect. Dis. (2020). doi:10.1016/S1473-3099(20)30327-3
30. Breidenstein, E. B. M., de la Fuente-Núñez, C. & Hancock, R. E. W. Pseudomonas aeruginosa: all roads lead to resistance. Trends Microbiol. 19, 419-426 (2011).
31. Pachori, P., Gothalwal, R. & Gandhi, P. Emergence of antibiotic resistance Pseudomonas aeruginosa in intensive care unit; a critical review. Genes Dis. 6, 109-119 (2019).
32. Tyers, M. & Wright, G. D. Drug combinations: a strategy to extend the life of antibiotics in the 21st century. Nat. Rev. Microbiol. 17, 141-155 (2019).
33. Reffuveille, F., de la Fuente-Núñez, C., Mansour, S. & Hancock, R. E. W. A Broad-Spectrum Antibiofilm Peptide Enhances Antibiotic Action against Bacterial Biofilms. Antimicrob. Agents Chemother. 58, 5363-5371 (2014).
34. Merg, F. et al. Big dynorphin as a putative endogenous ligand for the kappa-opioid receptor. J. Neurochem. 97, 292-301 (2006).
35. Wang, Z. et al. Elabela-Apelin Receptor Signaling Pathway is Functional in Mammalian Systems. Sci. Rep. 5, 8170 (2015).
36. Cheah, S.-E. et al. Polymyxin Resistance in Acinetobacter baumannii: Genetic Mutations and Transcriptomic Changes in Response to Clinically Relevant Dosage Regimens. Sci. Rep. 6, 26233 (2016).
37. Samaras, P. et al. ProteomicsDB: a multi-omics and multi-organism resource for life science research. Nucleic Acids Res. 48, D1153-D1163 (2019).
38. Fleming, A. Penicillin. Nobel Lecture (1945). Available at: https://www.nobelprize.org/uploads/2018/06/fleming-lecture.pdf. (Accessed: 7 Aug. 2020)
39. Fensterseifer, I. C. M. et al. Selective antibacterial activity of the cationic peptide PaDBS1R6 against Gram-negative bacteria. Biochim. Biophys. Acta—Biomembr. 1861, 1375-1387 (2019).
40. Cardoso, M. H. et al. A Computationally Designed Peptide Derived from Escherichia coli as a Potential Drug Template for Antibacterial and Antibiofilm Therapies. ACS Infect. Dis. 4, 1727-1736 (2018).
41. Cândido, E. S. et al. Short Cationic Peptide Derived from Archaea with Dual Antibacterial Properties and Anti-Infective Potential. ACS Infect. Dis. 5, 1081-1086 (2019).
42. Pane, K. et al. Identification of Novel Cryptic Multifunctional Antimicrobial Peptides from the Human Stomach Enabled by a Computational-Experimental Platform. ACS Synth. Biol. 7, 2105-2115 (2018).
43. Ling, L. L. et al. A new antibiotic kills pathogens without detectable resistance. Nature 517, 455-459 (2015).
44. Gudmundsson, S. & Erlendsdóttir, H. Murine Thigh Infection Model. in Handbook of Animal Models of Infection 137-144 (Elsevier, 1999). doi:10.1016/B978-012775390-4/50154-8
45. Sberro, H. et al. Large-Scale Analyses of Human Microbiomes Reveal Thousands of Small, Novel Genes. Cell 178, 1245-1259.e14 (2019).
46. Donia, M. S. & Fischbach, M. A. Small molecules from the human microbiota. Science (80-.). 349, 1254766-1254766 (2015).
47. Sugimoto, Y. et al. A metagenomic strategy for harnessing the chemical repertoire of the human microbiome. Science (80-.). 366, eaax9176 (2019).
48. Culp, E. J. et al. Hidden antibiotics in actinomycetes can be identified by inactivation of gene clusters for common antibiotics. Nat. Biotechnol. 37, 1149-1154 (2019).
49. The UniProt Consortium. UniProt: a worldwide hub of protein knowledge. Nucleic Acids Res. 47, D506-D515 (2019).

1. A method for computationally identifying candidate encrypted peptides with predicted antimicrobial activity comprising:
i) identifying a peptide segment of a first protein sequence from the human proteome, the segment being defined by a first sequence window of 5 to 80 amino acids in length, wherein the first residue within the sequence window represents the first residue within the protein sequence;
ii) assigning the identified peptide segment a score based on a scoring function that determines mean hydrophobicity, net charge, and length of the peptide, increases the determined mean hydrophobicity by 0.9, increases the determined net charge by 1.1, and multiples all obtained values linearly;
iii) identifying a further peptide segment of the protein sequence, the further peptide segment also being defined by the first sequence window length, but wherein the first residue within the sequence window for the further peptide segment represents the second residue within the protein sequence;
iv) assigning the further identified peptide segment a score based on said scoring function;
v) conducting additional scans of the protein sequence until all segments defined by the sequence window have been identified, and assigning each of the identified segments a score based on the scoring function;
vi) ranking all identified segments by score; and,
vii) repeating each of steps i)-vi) for all protein sequences from the human proteome;
wherein the segments that are ranked among the top 1,000 using the scoring function represent the candidate encrypted peptides for the first sequence window length.

2. The method according to claim 1, comprising repeating each of steps i)-vii) for a second sequence window length of 5 to 80 amino acids that is different from the first sequence window length, wherein the segments that are ranked among the top 1,000 using the scoring function represent the candidate encrypted peptides for the second sequence window length.

3. The method according to claim 1, comprising repeating each of steps i)-vii) for at least one other possible sequence window length from 5 to 80 amino acids.

4. The method according to claim 1, comprising repeating each of steps i)-vii) for every other possible sequence window length from 5 to 80 amino acids.

5. The method according to claim 1, wherein said ranking process of step vi) excludes from the ranking consecutive segments having a sequence overlap of about 50% or more.

6. The method according to claim 1, wherein the protein sequences from the human proteome includes all protein sequence isoforms from the human proteome.

7. The method according to claim 1, comprising performing step i) with respect to a second protein sequence at substantially the same time as performing step i) with respect to the first protein sequence, using the same sequence window of 5 to 80 amino acids.

8. A peptide, wherein said peptide is any one of SEQ ID NOS:1-43055.

9. A method of treating a microbial infection comprising administering to a subject in need thereof a pharmaceutically effective amount of a peptide of any one of SEQ ID NOS:1-43055.

10. The method according to claim 9, wherein the microbial infection is bacterial.

11. The method according to claim 10, wherein the microbial infection includes Escherichia coli ATCC11775, Pseudomonas aeruginosa PAO1, Pseudomonas aeruginosa PA14, Staphylococcus aureus ATCC12600, Escherichia coli AIG221, E. coli AIG222, Klebsiella pneumoniae ATCC133883, and Acinetobacter baumannii ATCC19606, or any combination thereof.

12. The method according to claim 9, comprising administering to the subject a pharmaceutically effective amount of a peptide of any one of SEQ ID NOS:43001-43055.

13. The method according to claim 12, comprising administering to the subject two or more peptides of SEQ ID NOS:43001-43055.

14. The method according to claim 13, comprising administering to the subject a combination of any two or more of SEQ ID NO: 43016, SEQ ID NO: 43015, natriuretic peptide A, SEQ ID NO: 43014, SEQ ID NO: 43024, SEQ ID NO: 43032, SEQ ID NO: 43040, SEQ ID NO: 43039, SEQ ID NO: 43041, SEQ ID NO: 43033, SEQ ID NO: 43034, and SEQ ID NO: 43035.

15. The method according to claim 13, comprising administering to the subject a peptide of SEQ ID NO: 43033 and a peptide of SEQ ID NO: 43035.
